(19) United States
(12) Patent Application Publication
(10) Pub. No.: US 2019/0117804 A1
(43) Pub. Date: Apr. 25, 2019

(54) Method for sanitizing biomass

(71) Applicant: CEBCON Technologies GmbH, Hamburg (DE)

(72) Inventor: Valery Detzel, Hamburg (DE)

(73) Assignee: CEBCON Technologies GmbH, Hamburg (DE)

(21) Appl. No.: 16/093,052

(22) PCT Filed: Mar. 24, 2017
(86) PCT No.: PCT/EP2017/057106

§ 371 Date: Oct. 11, 2018

(30) Foreign Application Priority Data
Apr. 12, 2016 (DE) 10 2016 106 746.6

(51) Int. Cl. A61L 2/04; C12M 1/107; C12M 1/00

(52) U.S. Cl. CPC A61L 2/04; C12M 21/04; C05F 3/00; C12M 47/20; C12M 47/16; C12M 23/58

(57) ABSTRACT

A method for sanitizing biomass in which the biomass is fed to a shaft cooler, and the biomass is heated in the shaft cooler by supplying a heated heating medium to the shaft cooler.

### BACKGROUND OF THE INVENTION

The invention relates to a method for sanitizing biomass.

### BRIEF SUMMARY OF THE INVENTION

Sanitation involves animal and human hygiene (killing human and animal pathogens) and phytohygiene (killing phytopathogens, quarantine pests, plant pests, fungi and viruses, and weed seeds). The aim of sanitation is to kill enough microorganisms that have a pathogenic effect on plants, animals and humans in biomasses so that only a minimum risk of transmitting diseases exists. By sanitizing these materials, epidemiological safety is achieved.

In light of the many uses, biomasses must undergo prior sanitation. In sanitation, harmful germs, or respectively microorganisms are killed. Examples of this are the use of chicken dung or manure as fertilizer, or the use of waste from agriculture, the timber industry and forestry as bedding, fermentation substrate for biogas systems, or other purposes in which a low bioburden is important.

Sanitation is carried out by exposing the biomass over a time period of for example at least one hour to an elevated temperature of for example 70° C. The conditions for sanitizing animal byproducts not intended for human consumption are presented in the directive (EC) No. 1069/2009 of Oct. 21, 2009.

It is known to perform sanitation in a stirring tank heated from the outside.

Sanitation is carried out in batches so that there is no backmixing, and so that unsanitized parts of the biomass are not further processed. Stirring is energy-intensive, and evenly heating the biomass is difficult to achieve. Batchwise operation only permits comparatively low volume throughputs.

Against this background, the object of the invention is to provide a method for sanitizing biomass that is improved in terms of energy with respect to even heating and volume throughputs.

In the method for sanitizing biomass according to the invention, the biomass is fed to a shaft cooler, and the biomass is heated in the shaft cooler by supplying a heated heating medium to the shaft cooler.

In the method according to the invention, a shaft cooler which is conventionally used for cooling pellets or other solid granules consisting of small particulate material of an organic and/or vegetable origin is used to sanitize biomass. For this, a heating medium instead of a cooling medium is supplied to the shaft cooler. The heating medium is supplied heated to the cooling medium inlet of the shaft cooler and discharged cooled from the cooling medium outlet of the shaft cooler. It is supplied to the cooling medium inlet at a temperature that is at least the same as the temperature for sanitizing the biomass. Preferably, pourable biomass is sanitized according to the method. The particularly effective and even transfer of heat from the heating medium to the biomass in the shaft cooler is advantageous due to the large heat-transferring surfaces which have contact with the biomass. It is moreover advantageous that the shaft cooler has a narrow dwell-time spectrum so that the material to be sanitized can travel evenly through the shafts. Consequently, sanitation by means of the shaft cooler can be continuous without material that is incompletely sanitized leaving the shaft cooler due to backmixing or a short-circuiting. In the continuous mode of operation, the biomass to be sanitized is continuously fed and discharged. This yields a particularly economical mode of operation. It is, however, in principle also possible to use the shaft cooler to sanitize biomass in batches by first filling the shaft cooler with the biomass to be sanitized, then sealing it and removing the sanitized material after a certain time period.

According to an advantageous embodiment of the invention, the biomass is of animal origin and/or vegetable origin.

According to another embodiment, the biomass is selected from at least one of the following materials: Chicken dung, manure or other animal excrements, slaughtering waste, animal cadavers or other animal wastes, food wastes, kitchen wastes or other household garbage components, sawdust or wood shavings, wood chips, wood shredder material or other byproducts or waste from the timber industry and forestry, straw, sunflower shells, olive pits, olive pressing residue, rice husks or other biological residue from agriculture or the food industry, fermentation substrate (feed substrates) for supplying biogas plants, or fermentation residue from biogas plants.

According to a preferred embodiment, the biomass is fed to the shaft cooler in pourable form, preferably in solid form, preferably in the form of pellets or other solid granules of small particulate material. The shaft cooler is particularly advantageous for heating the biomass in the form of pellets or other solid granules.

According to another embodiment, the biomass is heated in the shaft cooler to a temperature of at least 110° C., preferably at least 90° C., preferably at least 80° C., preferably at least 75° C., preferably at least 70° C., and/or the biomass is heated in the shaft cooler over a time span of at least 1.5 hours, preferably at least 1.1 hours, preferably at least 1 hour.

According to another embodiment, water or thermal oil is used as the heating medium to heat the biomass in the shaft cooler.

According to another preferred embodiment, a shaft cooler is used for sanitizing which is arranged at least partially in at least one container. Preferably, a shaft cooler is used that is completely arranged in a single container. This facilitates setting up a plant for sanitizing biomass, in particular when the plant is to be used at different times at different locations. The container is economically transportable on rails, by roadway or water from the plant to the site of use, or from an earlier site of use to a new site of use.

According to another embodiment, the container has the dimensions of a 20-foot or a 40-foot container, or another standard container. This is advantageous for transporting the container using available means of transportation. According to one preferred embodiment, the container has the features (such as stackability, transportability, lashing to each other) of standard containers. According to another embodiment, the container is a frame structure with open walls or one or more closed walls. The frame structure has a frame consisting of a plurality of frame parts. When there is an open frame structure, frame parts are surrounded by openings that are not filled with walls. When there is a closed frame structure, the openings between the frame parts are filled, or respectively closed by walls. Mixed forms with a partially open and partially closed frame structure are also a component of the invention. The frame structure is preferably cuboid, wherein the frame parts define the edges of the cuboid.

The design of the frame structure reduces weight and cost. In addition, it makes it easier to assemble, operate and service the plant.

Alternatively, the container is a conventional, standard container with closed walls. According to one configuration, the container is formed by walls connected to each other at the edges. The walls and/or the connections of adjacent walls with each other may be reinforced at the corners and/or edges of the container.

When the container is designed as a frame structure with open walls, the frame parts form the container shell. When the container is designed as a frame structure with one or more closed walls, the frame parts and the walls that fill them in form the container shell. When the container is designed as a box consisting of the walls connected to each other at the edge, the walls form the container shell.

According to another embodiment, at least one structural element of the shaft container is at least partially a component of a container shell. According to one preferred configuration, at least one structural element of the shaft cooler is a component of at least one wall and/or at least one frame part of the frame. According to another embodiment, at least one side wall and/or at least one frame part of the shaft cooler is a component of a wall and/or a frame part of the container. For example, at least one side wall of a cooler housing of the shaft cooler which borders a room in which the biomass to be cooled is cooled is a component of a wall of the container, or forms the entire wall of the container. Preferably, a plurality of side walls of the shaft cooler are simultaneously walls of the container, or components thereof.

According to another embodiment, the shaft cooler is accommodated in a container with a vertically oriented longitudinal axis.

According to another embodiment, the shaft cooler comprises a plurality of pipe meanders that are arranged parallel at a distance from each other and are offset in height by one-half the distance of their horizontal pipe sections. The pipe meanders are preferably oriented vertically. The material is alternately deflected horizontally in one direction and the other direction by the horizontal pipe sections of the pipe meanders while passing through the shafts of the shaft cooler formed between the pipe meanders. The shaft cooler heats the biomass particularly effectively and sparingly (in particular avoiding the formation of dust and large amounts of exhaust air) and has a small footprint and space requirement.

According to another embodiment, the horizontal pipe sections of each pipe meander are connected to each other by vertical bars. The vertical bars improve the stability of the pipe meander. In addition, they cause the material to be heated to be conducted in a manner advantageous for even heating and enlarge the heat exchanger surface.

The pipe meanders are for example formed from bent pipes between which bars are inserted. According to another configuration, the pipe meanders and bars are formed from metal plates that are shaped (for example by deep-drawing processes or hydroforming processes) and connected to each other analogous to known panel radiators made of plate steel.

According to another embodiment, the shaft cooler has a discharge floor at the bottom with an adjustable opening cross-section to regulate the fill level of the material within the shafts, and/or the shaft cooler has a feeding apparatus at the top for evenly distributing material to the various shafts. The shaft cooler can be operated particularly effectively by means of the discharge floor and/or the feeding apparatus.

According to another embodiment, the shaft cooler is connected to an apparatus for exhausting vapors and residual steam.

According to another embodiment, the shaft cooler is connected to an apparatus for exhausting vapors and residual steam, and heat recovery is integrated into the apparatus for exhausting. This can further improve the energy efficiency of the method.

According to another configuration, the sanitized biomass is fed to heat recovery. The heat recovered from the heated biomass can for example be used to preheat the biomass before being fed into the shaft cooler.

Due to its use for sanitizing according to the invention, the shaft cooler can also be termed a “shaft heater”.

### BRIEF DESCRIPTION OF THE SEVERAL VIEWS OF THE DRAWING

The invention is explained in greater detail below based on included drawings of an exemplary embodiment. In the drawings:

FIG. 1 shows a first vertical section of a shaft cooler;

FIG. 2 shows a second vertical section of the shaft cooler;

FIG. 3 shows a side view of the pipe meanders of the shaft cooler;

FIG. 4 shows a front view of a pipe meander of the shaft cooler;

FIG. 5 shows a front view of another pipe meander of the shaft cooler;

FIG. 6 shows an overview drawing of a shaft cooler (outside view);

FIG. 7 shows an overview drawing of a shaft cooler (inside view).

### DETAILED DESCRIPTION OF THE INVENTION

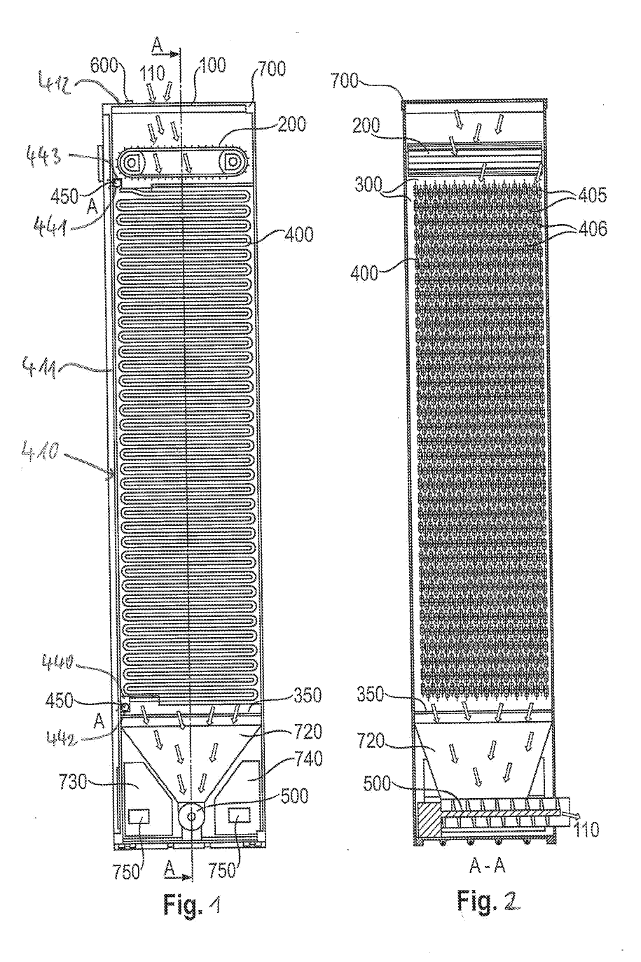

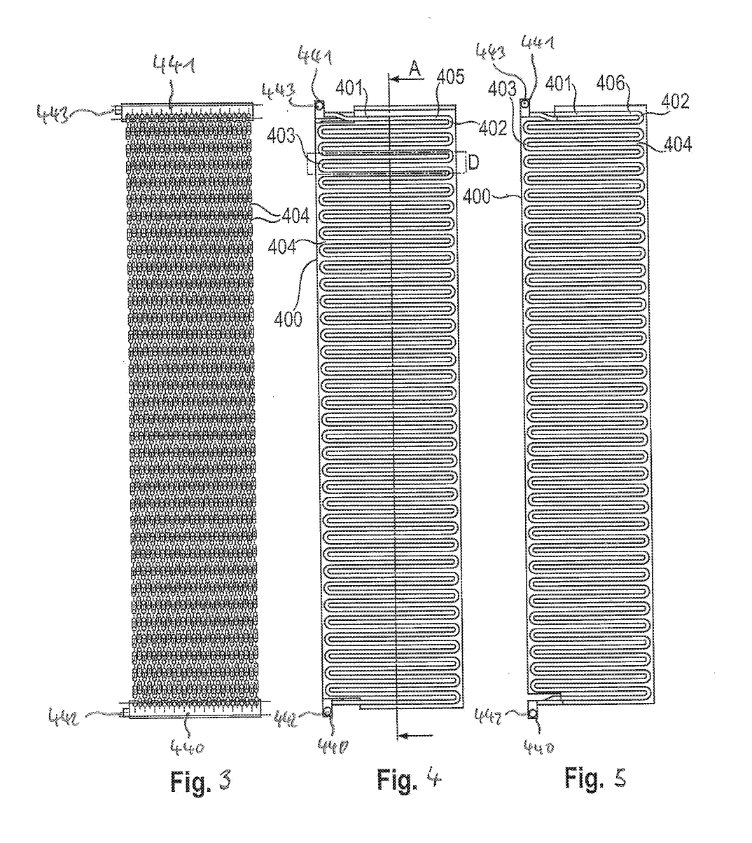

The configuration and functioning of the shaft cooler 12 will be explained with reference to FIGS. 1 to 7.

Pellets 110, granules or other pourable material consisting of biomass (hereinafter simply termed “pellets”) are introduced through the filling opening 100 in a cover wall 412 of a box-shaped cooler housing 410 and distributed with a drag chain conveyor 200 arranged below the cover wall over parallel vertical shafts 300 within four side walls 411 of the cooler housing 410. A movable slotted floor 350 below the shafts 300 remains closed until the shafts 300 are full. Heating medium 450 is guided through the heat exchanger body 400, and it supplies the pellets 110 with thermal energy. The pellets 110 are thereby heated to 70° C., for example.

The pellets 110 continuously and evenly slide downward in the shafts 300, wherein their speed is regulated by the gap size in the slotted floor 350. The speed is adjusted so that the dwell time of the pellets 110 in the shafts 300 is one hour.

After passing through the slotted floor 350, the heated pellets 110 are supplied by a discharge funnel 720 to a discharge screw 500 and are discharged therefrom. The discharge funnel 720 borders the bottom of the cooler housing 410. The openings 600 in the cover wall 412 serve to release the vapors and residual steam from the hot pellets 110. Furthermore, an inspection flap 416 is in the cover wall 412.

The design of the heat exchanger bodies 400 is shown in FIGS. 1 to 5.

The heat exchanger bodies 400 are designed as connected serpentine pipe systems. The heat exchanger bodies 400 have parallel, horizontal pipe sections 401 that are connected at the ends by pipe elbows 402 with which they form pipe meanders 403. Furthermore, they have vertical bars 404 between adjacent horizontal pipe sections 401.

Two groups 405, 406 of heat exchanger bodies 400 are installed, wherein the horizontal pipe sections 401 of the heat exchanger bodies 400 of different groups 405, 406 are offset from each other by one half of distance D between two adjacent, horizontal pipe sections 401 of a heat exchanger body 400. This causes a sideways movement of the pellets 110 in the shaft 300 and an enlargement of the contact surface. At the same time, there is a proportionate circulation of the pellets 110 in the shaft 300 which improves the evenness of the heating.

Each pipe meander 403 terminates at the bottom in a bottom manifold 440 and at the top in a top manifold 441. The bottom manifold 440 has a bottom opening 442 in a side wall 411 of the shaft cooler 12 which serves as a cooling medium inlet. The top manifold 441 has a top opening 443 at a top end in a side wall 411 of the shaft cooler 12 which serves as a cooling medium outlet. The heating medium 450 enters through the bottom opening 442 into the shaft cooler 12 and leaves therefrom through the top opening 443.

The shaft cooler is entirely accommodated in a horizontally or vertically aligned container.

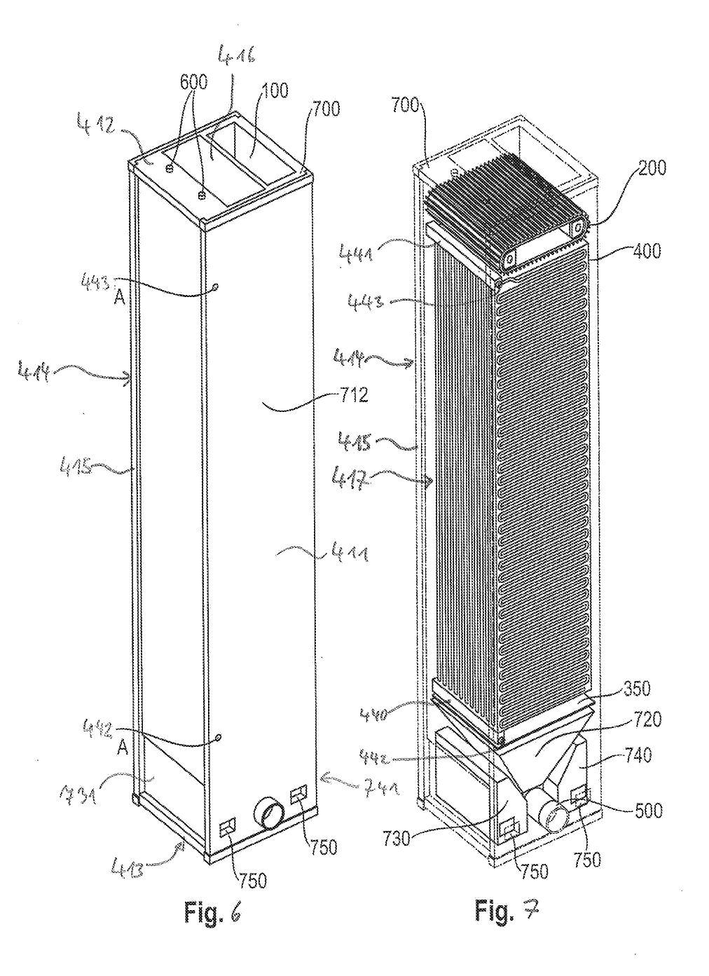

FIGS. 6 and 7 show a vertically oriented container 700 which contains a complete shaft cooler 712. The side walls 411 and the cover wall 412 of the cooler housing 410 are simultaneously outer walls of the container 700. The side walls 411 preferably extend to the bottom end of the container 700. The container 700 preferably has a floor wall 413 at the bottom. Preferably, the container 700 has a frame 414, and the side walls 411, cover wall 412 and floor wall 413 are each held by the edges in openings 417 between frame parts 415 of the frame 414 to form an at least partially closed frame. Due to the integral construction of the walls 411, 412 and frame parts 415 of the cooler housing 410 and the container 700, bearing structural elements of the container 700 are simultaneously components of the shaft cooler 12 which saves materials and weight.

According to FIGS. 1, 6 and 7, control cabinets 730, 740 are arranged in the spaces within the container 700 on both sides of a discharge funnel 720 that supplies the heated pellets to the discharge screw 500. Interfaces 750 for energy and data are located on the control cabinets 730 and 740. At that location, the walls of the container 700 preferably have further inspection flaps or inspection openings 731, 741.

### REFERENCE NUMBER LIST

12 Cooler
110 Pellets
200 Drag chain conveyor
300 Shaft
350 Slotted floor
400 Heat exchanger body
401 Horizontal pipe section
402 Pipe elbow
403 Pipe meander
404 Vertical bar
405 Group
406 Group
410 Cooler housing
411 Side wall
412 Cover wall
413 Floor wall
414 Frame
415 Frame part
416 Inspection flap
417 Opening
440 Bottom manifold
441 Top manifold
442 Bottom opening
443 Top opening
450 Heating medium
500 Discharge screw
600 Opening
700 Container
712 Shaft cooler
720 Discharge funnel
730 Control cabinet
731 Inspection opening
740 Control cabinet
741 Inspection opening
750 Interface for energy and data

1. A method for sanitizing biomass in which the biomass is fed to a shaft cooler, and the biomass is heated in the shaft cooler by supplying a heated heating medium to the shaft cooler.

2. The method according to claim 1, wherein the biomass is of animal origin and/or vegetable origin.

3. The method according to claim 2, wherein the biomass is selected from at least one of the following materials: Chicken dung, manure or other animal excrements, slaughtering waste, animal cadavers or other animal wastes, food wastes, kitchen wastes or other household garbage components, sawdust or wood shavings, wood chips, wood shredder material or other byproducts or waste from the timber industry and forestry, straw, sunflower shells, olive pits, olive pressing residue, rice husks or other biological residue from agriculture or the food industry, fermentation substrate for biogas plants, or fermentation residue from biogas plants.

4. The method according to one of claim 1, wherein pourable biomass, preferably in solid form, preferably in the form of pellets or other solid granules of small particulate material, is fed to the shaft cooler.

5. The method according to one of claim 1, wherein the biomass in the shaft cooler is heated to a temperature of at least 70° C., and/or wherein the biomass in the shaft cooler is heated over a time span of at least one hour.

6. The method according to one of claim 1, wherein water or thermal oil is used as the heating medium to heat the biomass in the shaft cooler.

7. The method according to one of claim 1, wherein the shaft cooler is arranged entirely or partially in at least one container.

8. The method according to one of claim 1, wherein the shaft cooler is at least partially arranged in at least one individual transportable container that has a container shell, wherein at least one structural element of the shaft container cooler is at least partially a component of the container shell.

9. The method according to one of claim 1, wherein the shaft cooler comprises a plurality of pipe meanders that are arranged parallel at a distance from each other and are offset in height by one half the distance of their horizontal pipe sections so that the material is alternately deflected horizontally in one direction and the other direction by the horizontal pipe sections of the pipe meanders while passing through the shafts of the shaft cooler formed between the pipe meanders.

10. The method according to claim 9, wherein the horizontal pipe sections of each pipe meander are connected to each other by vertical bars.

11. The method according to one of claim 1, wherein the shaft cooler has a discharge floor at the bottom with an adjustable opening cross-section to regulate the fill level of the material within the shafts, and/or a feeding apparatus at the top for evenly distributing material to the various shafts.
